(19) United States
(12) Patent Application Publication
(10) Pub. No.: US 2019/0060266 A1
(43) Pub. Date: Feb. 28, 2019

(54) PHARMACEUTICAL COMPOSITIONS OF TIOPRONIN AND METHODS FOR PREPARING THEREOF

(71) Applicant: Imprimis Pharmaceuticals, Inc., San Diego, CA (US)

(72) Inventor: Dennis Elias Saadeh, Irvine, CA (US)

(21) Appl. No.: 16/174,954

(22) Filed: Oct. 30, 2018

Related U.S. Application Data
(63) Continuation-in-part of application No. 15/383,211, filed on Dec. 19, 2016.
(60) Provisional application No. 62/271,020, filed on Dec. 22, 2015.
(60) Provisional application No. 62/272,894, filed on Dec. 30, 2015.

(51) Int. Cl. A61K 31/198; A61K 9/10; A61K 47/10; A61K 47/14; A61K 47/44; A61K 47/38; A61K 47/26; A61K 47/32; A61P 11/00; A61P 13/12

(52) U.S. Cl. CPC A61K 31/198; A61K 9/10; A61K 47/10; A61K 47/14; A61P 13/12; A61K 47/38; A61K 47/26; A61K 47/32; A61P 11/00; A61K 47/44

(57) ABSTRACT

Pharmaceutical compositions are described herein, the compositions consisting essentially of anhydrous suspensions of therapeutically effective quantity of a pharmaceutically acceptable reducing agent capable of undergoing thiol-disulfide exchange with cystine to form a mixed disulfide (such as tiopronin). In some embodiments, the suspension also contains one or more urine alkanizing agent(s). Methods for fabricating the compositions and using them are also described.

### CROSS REFERENCE TO RELATED APPLICATIONS

This application is a continuation-in-part application of U.S. application Ser. No. 15/383,211, filed Dec. 19, 2016, now pending, which claims priority under 35 U.S.C. § 119(e) to each of U.S. Provisional Application No. 62/271,020, filed Dec. 22, 2015, and to U.S. Provisional Application No. 62/272,894, filed on Dec. 30, 2015. The entire content of each of which is hereby incorporated by reference.

### FIELD OF THE INVENTION

The present invention relates generally to the field of pharmaceuticals, and, more specifically, to pharmaceutical compositions that include as an active component a therapeutically effective quantity of tiopronin, or a derivative or analog thereof, and to methods of preparing and using such compositions.

### BACKGROUND

Tiopronin, formally, 2-mercaptopropionylglycine (MPG), is a thiol drug that is commonly used to treat cystinuria, an inherited condition that occurs when there is too much of the amino acid cystine in the urine, leading to the formation of stones in the kidneys, bladder, and/or ureter. Cystine, an amino acid formed of two cysteine molecules via a disulfide bond, has limited solubility. Tiopronin, an acylated sulfhydryl-containing derivative of glycine, breaks the disulphide bond of cystine and binds the sulfhydryl group of the resultant cysteine monomers. This leads to a reduction in the concentrations of urinary cystine and reduces cystine stone formation.

The symptoms of cystinuria include strong intermittent or constant pain, hematuria, nausea, vomiting, and urinary urgency. In severe cases, nephrolithiasis can cause permanent kidney damage and even death. In pediatric patients, the incidence of urolithiasis (urinary tract calculi or stones) and nephrolithiasis (kidney calculi or stones) is on the rise due to dietary changes, metabolic abnormalities, and climate change, including in very young children that are pre-school-age. Although this is a less prevalent condition than in adults, its incidence should not be underestimated since the stones tend to recur and can be a cause of acute kidney injury (obstructive nephrolithiasis) in early childhood and significant morbidity.

THIOLA® (tiopronin) tablets are FDA-approved for prevention of cystinuria in patients with severe homozygous cystinuria with urinary cystine greater than 500 mg/day, and who were not successfully treated with dietary changes and increased fluid intake, or patients who have had side effects with the drug d-penicillamine. Dosing of THIOLA® for adults may be up to 1,000 mg/day administered in divided doses three time daily. For children 9 years of age or older, 15 mg/kg/day may be administered in three doses. However, THIOLA® is only available as 100-mg tablets. Patients must therefore take up to 10 tablets per day in 100-mg increments without options for precise, intermediary dosing.

Accordingly, there exists a need for improved compositions of tiopronin whereby more precise and customizable dosing can be achieved to reduce the cost of therapy, to reduce side effects, and to address difficulties patients may have in swallowing tablets. This patent specification discloses such pharmaceutical compositions of tiopronin, and methods of fabricating and administering the same in order to address the drawbacks of the existing formulations including allowance for precise and flexible dosing adjustments.

### SUMMARY

According to one embodiment of the invention, a pharmaceutical composition formulated as an anhydrous suspension is provided, the composition includes a dispersed phase comprising a therapeutically effective quantity of tiopronin, derivatives or analogs thereof, related cystine-binding thiol drugs, or tiopronin prodrugs, and an anhydrous dispersion medium, and may further include at least one pharmaceutically acceptable surfactant or solubilizing and suspending agent, wherein the dispersed phase is dispersed within the dispersion medium.

In various embodiments of the invention, the anhydrous dispersion medium includes at least one of a vegetable oil (e.g., castor oil, soybean oil, coconut oil, avocado oil, olive oil, almond oil) and a medium chain triglyceride.

In yet further embodiments of the invention, the acceptable surfactant or solubilizing and suspending agent may be any of non-ionic polyoxyethlene-polyoxypropylene block copolymers, a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, glyceryl distearate, triglycerol monooleate, and combinations thereof.

According to various further embodiments of the invention, the pharmaceutical compositions described herein may be orally administered to a mammalian subject in need of such treatment, to treat various diseases and maladies including, but not limited to, stones in the kidney, bladder, or ureter.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20, as well as to the numbers in between integers, e.g., 1.5 or 2.5, and the like.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “suspension” is defined for the purposes of the present application as a two-phase solid-in-liquid dispersion system having a first phase and a second phase. In other words, “suspension” is defined as a heterogeneous mixture in which the solute particles (i.e., those forming the solid phase) do not truly dissolve, but rather, are suspended throughout the bulk of the solvent, without undergoing any significant precipitation within prolonged periods of time. It is further specifically provided that dispersion systems having three, four or more phases are not within the meaning of “suspension” for the purposes of the instant application.

Therefore, the above mentioned first phase of the suspension consists of a multitude of solid particles and is designated and defined as the dispersed phase. The above mentioned second phase of the suspension is a liquid and is designated and defined as the dispersion medium, or, interchangeably and synonymously, the continuous phase.

Furthermore, the dispersed phase is dispersed in the dispersion medium, and the term “dispersed” is defined as meaning that the dispersed phase is statistically evenly distributed within the continuous phase throughout the entire volume of the suspension, with no statistically meaningful deviations in the concentrations of the dispersed phase in different portions of the suspension.

The term “medium-chain triglycerides” refers to triglycerides (i.e., tri-esters of glycerol and fatty acids) in which at least two of the three fatty acid moieties are derived from aliphatic (i.e., saturated open-chain) acids having between 6 and 12 carbon atoms; the fatty acids that are used for making medium-chain triglycerides are defined as medium-chain fatty acids and are caproic (IUPAC, hexanoic), enanthic (IUPAC, heptanoic), caprylic (IUPAC, octanoic), pelargonic (IUPAC, nonanoic), capric (IUPAC, decanoic), undecylic acid (IUPAC, undecanoic), or lauric (IUPAC, dodecanoic) acids.

The term “carrier” refers to a substance that serves as a vehicle for improving the efficiency of delivery and the effectiveness of a pharmaceutical composition.

The term “solubilizing agent” for the purposes of the instant application refers broadly to chemical compounds that improve the process of incorporating the solubilizate (i.e., active components described herein) into micelles. In other words, the presence of a solubilizing agent makes the process of solubilization faster, easier, and/or more complete as compared with compositions without it.

The term “suspending agent” used herein interchangeably with the term “emulsifier” for the purposes of the instant application refers broadly to chemical compounds that help active pharmaceutical ingredients stay suspended in the formulation and prevent and/or reduce the phase separation of the two-phase dispersion systems described herein.

The term “alkanizing agent” refers to a chemical compound or drug that is administered to a patient having diseases or medical disorders associated with low pH of bodily fluids (e.g., blood), in order to increase the pH thereof.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutical composition” is defined as a chemical or biological compound or substance, or a mixture or combination of two or more such compounds or substances, intended for use in the medical diagnosis, cure, treatment, or prevention of disease or pathology.

The term “pharmaceutically acceptable” when used in reference to a carrier, whether diluent or excipient, refers to a carrier that is compatible with the other ingredients of the formulation and not deleterious to the recipient thereof.

The terms “administration of a composition” or “administering a composition” are defined to include the act of providing a compound of the invention or pharmaceutical composition to a subject in need of treatment.

The terms “kidney stone disease” and “nephrolithiasis” refer to a urological or nephrological disease or condition manifesting itself by having renal calculi (nephroliths) formed and deposited in the patient's kidneys.

The terms “bladder stone disease” and “ureter stone disease” refer to urological diseases or conditions manifesting themselves by having stone-like matter (cystoliths) formed and deposited in the patient's urinary bladder or ureter, respectively.

The term “reducing agent” refers to an electron-donor compound, i.e., a compound that donates an electron to another chemical species in a redox chemical reaction.

The term “thiol” refers to an organic compound that is a sulfur-containing analog of an alcohol, i.e., a compound containing the group —SH.

The term “thiol disulfide exchange” refers to a chemical reaction described generally as follows:

RS—SR+R′SH [symbol] R'S—SR+RSH,

wherein each of R and R′ is an organic radical.

The term “amino acid” refers to an organic compound having both a carboxyl (—COOH) and an amino (—NH₂) group.

The term “glycine” refers to an aminoacetic acid having the structure NH₂—CH₂—COOH; and the term “cystine” refers to 2-amino-3-(2-amino-2-carboxyl-ethyl)disulfanylpropanoic acid (i.e., an amino acid having the structure HOOC—CH(NH₂)—S—S—CH₂—CH(NH₂)—COOH).

### B. Embodiments of the Invention

According to embodiments of the present invention, pharmaceutical compositions intended to prevent and/or treat various diseases and maladies including kidney stone disease, bladder stone disease or ureter stone disease are provided.

According to one embodiment of the invention, a pharmaceutical composition for treating, mitigating or preventing nephrolithiasis or urolithiasis is provided. It is further specifically provided that the compositions of the invention are formulated as suspensions.

According to one embodiment, the composition comprises a therapeutically effective quantity of at least one pharmaceutically acceptable reducing agent capable of undergoing thiol-disulfide exchange with cystine to form a mixed disulfide.

In some embodiments, the reducing agent comprises a thiol moiety and an amino acid moiety and may be, e.g., N-(2-mercaptopropionyl) glycine having the chemical formula:

CH₃—CH(SH)—C(O)—NH—CH₂—COO,

also known as tiopronin, or under the trade name THIOLA® (Mission Pharmacal Co., San Antonio, Tex.). Tiopronin is capable of binding cystine by thiol-disulfide exchange to form a mixed disulfide of tiopronin-cysteine.

Alternatively, (2S)-2-amino-3-methyl-3-sulfanylbutanoic acid, also known as D-penicilamine or under the trade name CUPRIMINE® (Valeant Pharmaceuticals International, Inc., Laval, Quebec, Canada) and having the formula:

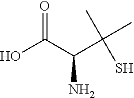

may also be used as the pharmaceutically acceptable reducing agent capable of undergoing thiol-disulfide exchange with cystine to form a mixed cysteine-containing disulfide. In various embodiments, penicilamine may be used as the sole reducing agent in the composition or in a combination with tiopronin, if desired.

Another example of a reducing agent that may be used in addition to, or instead of, tiopronin and/or penicilamine is captopril (1-(3-mercapto 2-methyl-1-oxopropyl)-L-proline), known under the trade name CAPOTEN® (Bristol-Myers Squibb).

Those having ordinary skill in the art may use (an)other reducing agent capable of undergoing thiol-disulfide exchange with cystine to form a mixed disulfide instead of, or in combination with, the above-named compound(s), if desired. In various embodiments, the concentration of the reducing agent(s) described above, in the compositions, may be between 1.0% mass % and 5.0 mass % of the total units of the suspension (weight/volume), for example, 1 mass %, 2 mass %, 3 mass %, or 4 mass %.

According to various embodiments, the composition comprising a therapeutically effective quantity of at least one pharmaceutically acceptable reducing agent capable of undergoing thiol-disulfide exchange with cystine to form a mixed disulfide may include a second active component consisting of a therapeutically effective quantity of a urine alkanizing agent such as potassium citrate, sodium citrate, magnesium citrate, sodium bicarbonate or combinations thereof may be used. In various embodiments, the concentrations of the urine alkanizing agent(s) in the compositions may be between 2 mass % and 20 mass %, for example, 4 mass %, 6 mass %, 8 mass %, 10 mass %, 12 mass %, 14 mass %, 16 mass %, or 18 mass %.

As disclosed herein, the compositions are in the form of a suspension. The suspensions include, consist of, or consist essentially of, an anhydrous dispersion medium (i.e., the continuous phase), a dispersed phase that is dispersed within the dispersion medium, and a pharmaceutically acceptable carrier. The dispersed phase includes, consists of, or consists essentially of, particles of a therapeutically effective quantity of at least one pharmaceutically acceptable reducing agent capable of undergoing thiol-disulfide exchange with cystine to form a mixed disulfide, or derivatives or analogs thereof. The dispersion medium includes at least one of a vegetable oil or a medium chain triglyceride, and further comprises at least one emulsifier or solubilizing and suspending agent.

As mentioned above, the anhydrous dispersion medium of the composition of the present invention is comprised of at least vegetable oil or at least one medium chain triglyceride or a combination of products of both classes. In various embodiments, the dispersion medium forms the major portion of the composition, its mass concentration in the composition being between about 80.0 mass % and about 99.0 mass %, such as between about 85.0 mass % and about 95.0 mass %, for example, about 90 mass %, 92.5 mass %, or 95.0 mass %.

Specific examples of acceptable vegetable oils that may be used in the anhydrous dispersion medium according to various embodiments of the invention include, without limitation, castor oil, soybean oil, coconut oil, avocado oil, olive oil, almond oil, and combinations thereof. Those having ordinary skill in the art may use (an)other vegetable oil(s) instead of, or in combination with, those mentioned above, if desired. Those having ordinary skill in the art will understand that all these oils represent complex blends of organic compounds, as opposed to individual organic molecules.

For example, castor oil is a complex mixture of several fatty acids, principally, ricinoleic acid, an unsaturated, 18-carbon fatty acid having a hydroxyl functional group on the 12^{th} carbon (IUPAC, 12-hydroxyoctadec-9-enoic acid). Those having ordinary skill in the art will understand that castor oil has a very complex chemical structure and is a mixture of triglycerides that varies, but commonly comprises ricinoleic acid (about 70%) plus triglycerides of linoleic (IUPAC, 9,12-octadecadienoic) and oleic (IUPAC, octadec-9-enoic) acids (about 20% combined).

Almond oil is another complex mixture of several fatty acids, which varies but typically comprises 65 to 70 mass % of oleic, 20 to 25% of linoleic, up to 4% of palmitic (IUPAC, hexadecanoic) and small quantities of palmitoleic (IUPAC, hexadec-9-enoic) and stearic (IUPAC, octadecanoic) acids.

Coconut oil is yet another complex mixture of several fatty acids, which also varies, but commonly comprises about 45 to 50% of lauric acid, the balance being a combination of other medium-chain fatty acids described above, as well as palmitic and stearic acids.

Olive oil is yet another mixture of several fatty acids, which also varies, but its principal ingredient is oleic acid (about 75 to 85%), the balance being a combination of other fatty acids including linoleic and palmitic acids.

In various embodiments, a variety of medium-chain triglycerides can be used for forming the anhydrous dispersion medium of the compositions of the invention. For example, triglyceride(s) containing the aliphatic tails derived from caprylic acid or caproic acid may be so used. Those having ordinary skill in the art may use (an)other medium-chain triglyceride(s) instead of, or in combination with, those based on caprylic or caproic acids, if desired. One specific product comprising medium-chain triglycerides that may be used is UNISPEND® anhydrous sweetened liquid (Fagron, Inc., St. Paul, Minn.). As stated above, the anhydrous dispersion medium used herein further comprises at least one emulsifier or solubilizing and suspending agent which may be present in the compositions of the instant invention at mass concentrations between about 0.1 mass % and about 10.0 mass %, such as between about 1.0 mass % and about 5.0 mass %, for example, about 1.0 mass %, 2.0 mass %, 3.0 mass % or 4.0 mass %.

Those having ordinary skill in the art will choose the most appropriate emulsifier or solubilizing and suspending agent. For example, one such emulsifier or solubilizing and suspending agent that may be used is a non-ionic polyoxyethlene-polyoxypropylene block copolymer having the general structure HO—(CH₂—CH₂—O)ₓ—(C₃H₆—O)_{y}—(CH₂—CH₂—O)ₓ—H, wherein x is an integer having the value of at least 8, and y is an integer having the value of at least 38. Polyoxyethlene-polyoxypropylene block copolymer(s) that can be used may be those belonging to the PLURONIC® or POLOXAMER® families, chemically, poly(ethylene glycol)-block-poly(propylene glycol)-block-poly(ethylene glycol), both available from BASF Corp. and from several other vendors and having the following general chemical structure:

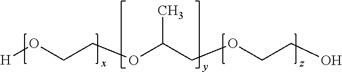

A specific and non-limiting example of a non-ionic polyoxyethlene-polyoxypropylene block copolymer that can be used is the product known under the trade name PLURONIC® L64, which is described by the chemical structure above, with the molecular weight of the polyoxypropylene portion of about 1,750 Daltons, about a 40% polyoxyethylene content (mass), and the average overall molecular weight of about 2,900 Daltons. Another specific non-limiting example of a non-ionic polyoxyethlene-polyoxypropylene block copolymer that can be used is the product known under the trade name POLOXAMER 407® (also known as PLURONIC® F127), which is also described by the chemical structure above, with the molecular weight of the polyoxypropylene portion of about 4,000 Daltons, about a 70% polyoxyethylene content (mass), the overall molecular weight of between about 9,840 Daltons and about 14,600 Daltons.

Some non-limiting examples of other emulsifiers or solubilizing and suspending agents that may be used in combination with, or instead of, non-ionic polyoxyethlene-polyoxypropylene block copolymers, include derivatives of cellulose, optionally partially cross-linked polyacrylates, polyoxyethylene sorbitan monolaurates, glyceryl isostearate, polyoxyethylene sorbitan monopalmitates, polyoxyethylene sorbitan monostearates, polyoxyethylene sorbitan monooleates (e.g., members of POLYSORBATE® family of products), glyceryl distearate, triglycerol monooleate, and polysaccharide thickening agents such as xanthan gum.

For example, suitable derivatives of cellulose that may be used include, without limitations, carboxymethyl cellulose, methyl cellulose, hydroxyethyl cellulose, and hydroxypropyl cellulose (Dow Chemical, Midland, Mich.). Examples of acceptable partially cross-linked polyacrylates that may be used include, without limitations, polymers of the CARBOPOL® family (Lubrizol, Wickliffe, Ohio). Typically, the cross-linking agents that may be used to cross-link such polyacrylates are allyl sucrose or allyl pentaerythritol.

Suitable products of the POLYSORBATE® family (i.e., ethoxylated sorbitan esterified with fatty acids) that may be used include, without limitations, polyoxyethylene sorbitan monolaurates, polyoxyethylene sorbitan monopalmitates, polyoxyethylene sorbitan monostearates, or polyoxyethylene sorbitan monooleates, some of which are also known as TWEEN® products, such as POLYSORBATE® 80) (Croda, Wilmington, Del.).

One typical product of the latter family that can be used is POLYSORBATE 80® (chemically, polyoxyethylene (20) sorbitan monooleate, also known as sorbitan mono-9-octadecenoate poly(oxy-1,2-ethanediyl), i.e., a product of polycondensation of polyethoxylated sorbitan and oleic acid having 20 units derived from ethylene glycol), which is a nonionic surfactant and emulsifier having the structure.

In various embodiments, the pharmaceutical composition may further optionally include one or several pharmaceutically acceptable excipient(s). In some embodiments, an excipient that can be used may be one or several filler(s) to be selected by those having ordinary skill in the art, such as polyacrylate dispersion, e.g., Eudagrit NE 30 D® (available from Evonik Industries, Parsippany, N.J.), which is a component allowing delayed or controlled release. Such excipients can be used for preparing the formulations in the form of a suspension to protect from gastric acid and delay pH dependent dissolution. Therefore, in some embodiments, formulations may be optionally compounded as delayed release compositions. The concentration of such excipient(s), if used, in the compositions may between about 50.0 mass % and about 80.0 mass % of the total mass of the suspension.

In various embodiments, excipients that can be used for fabricating the pharmaceutical compositions described herein may optionally include one or more of various taste modifiers such as sweeteners (e.g., sucrose and derivatives, sodium saccharin, aspartame, stevioside, monosodium glycyrrhizinate), flavoring agents (e.g., those introducing any natural or artificial fruit, vegetable, flower, beverage or candy flavor, such as cherry, citrus (lemon, orange, tangerine), raspberry, vanillin and vanilla, marshmallow, chocolate, etc.), or anesthetic agents (e.g. menthol, peppermint, cinnamon).

Those having ordinary skill in the art will realize that yet (an)other additional emulsifier(s) or solubilizing and suspending agent(s) may be used, if desired, and will select such supplemental emulsifier(s) or solubilizing and suspending agent(s), as well as to choose the quantity thereof.

According to various embodiments of the present application, the pharmaceutical compositions described herein are formulated as stable two-phase suspensions as defined above. More specifically, according to these embodiments, the suspensions consist of two phases, i.e., the dispersed phase that is dispersed within the dispersion medium. The dispersed phase includes particles comprising a therapeutically effective quantity of the pharmaceutically active component, i.e., tiopronin or another pharmaceutical composition containing an agent capable of binding cystine by thiol-disulfide exchange. In some embodiments, no compounds other than tiopronin or its derivatives or analogs as described hereinabove are present within the particles that form the dispersed phase. According to such embodiments, the dispersion medium is a liquid that includes all other compounds that are present in the pharmaceutical compositions described in the application. The application therefore envisions no embodiment where tiopronin can be used outside the dispersed phase, such as being a part of the dispersion medium.

In various embodiments, in addition to tiopronin or derivatives or analogs thereof, the dispersed phase may optionally contain other compounds, such as, without limitation, stabilizers, anti-oxidants, preservatives, various flavoring agents or sweeteners.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Those having ordinary skill in the art can choose the best method for preparing the compositions.

Pharmaceutical formulations described herein can typically be administered orally. An ordinarily skilled physician may prescribe delivery by any other acceptable method if so desired and indicated.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, diet, and the severity of the particular disease or condition being treated.

The medication prepared as described above may then be prescribed and given to a patient for treating, mitigating or preventing kidney stone disease, bladder stone disease or ureter stone disease. Among various kinds of kidney, bladder or ureter stone disease that may be treated, one kind of treatment that is particularly envisioned according to embodiments of the present invention is the treatment, mitigation or prevention of cystinuria. Non-limiting examples of other diseases or conditions that may be treated using the compositions described herein include rheumatoid arthritis and mucolytic treatments (i.e., treatments for clearing of mucus from the airways, lungs, bronchi, and trachea of a patient).

### C. Examples

### Example 1. Preparing a Pharmaceutical Composition No. 1

A pharmaceutical composition was prepared as described below. The following products were used in the amounts specified:
(a) about 4.0 g of solid powdered tiopronin;
(b) about 0.1 g of glyceryl isostearate;
(c) about 0.2 mL of POLYSORBATE® 80;
(d) enough UNISPEND® anhydrous sweetened medium chain triglyceride to make a final volume of 100 mL;
(e) about 1.5 mL of artificial caramel flavor liquid; and
(f) a small quantity of glycerol sufficient to wet the powders, as mentioned below.

Tiopronin was triturated using a mortar and pestle according to standard techniques of mixing solids to achieve uniformity. A small quantity of glycerol was added to wet the powder followed by trituration again to form a smooth paste.

The artificial caramel flavor liquid and POLYSORBATE® 80 were then added with additional trituration, followed by adding the anhydrous sweetened medium chain triglyceride. The product was then transferred to the dispensing bottle. Finally, the mortar was washed using a small quantity of anhydrous sweetened medium chain trigylceride, and the wash was transferred to the bottle, to ensure the entire quantity of active components has been so transferred, followed by packaging and labeling.

### Example 2. Preparing a Pharmaceutical Composition No. 2

A pharmaceutical composition was prepared as described below. The following products were used in the amounts specified:
(a) about 4.0 g of tiopronin powder;
(b) about 21.6 g of potassium citrate powder;
(c) about 0.1 g of glyceryl isostearate;
(d) about 50 mL of Eudacrit NE 30 D®;
(e) enough 100 mL of UNISPEND® anhydrous sweetened medium chain triglyceride to make a final volume of 100 mL;
(f) about 1.5 mL of artificial caramel flavor liquid; and
(g) a small quantity of glycerol sufficient to wet the powders, as mentioned below.

Tiopronin and potassium citrate powders were mixed using a mortar and pestle method by using the principles of trituration and geometric dilution known to those having the skill in the art of preparing pharmaceutical compositions.

A small quantity of glycerol was added to wet the powder followed by trituration again to form a smooth paste. The artificial caramel flavor liquid and POLYSORBATE® 80 were then added, with trituration followed by addition of Eudacrit NE 30 D® and the anhydrous sweetened medium chain triglyceride with mixing. The product was the transferred to the dispensing bottle. Finally, the mortar was washed using a small quantity of anhydrous sweetened medium chain triglyceride, and the wash was then transferred to the bottle to ensure the entire quantity of active components has been so transferred followed by packaging and labeling.

Although the invention has been described with reference to the above examples, it will be understood that modifications and variations are encompassed within the spirit and scope of the invention. Accordingly, the invention is limited only by the following claims.

What is claimed is:

1. A pharmaceutical composition formulated as an anhydrous suspension comprising:
(a) a dispersed phase comprising a therapeutically effective quantity of a pharmaceutically acceptable reducing agent capable of undergoing thiol-disulfide exchange with cystine to form a mixed disulfide;
(b) at least one pharmaceutically acceptable surfactant or solubilizing and suspending agent; and
(c) an anhydrous dispersion medium,
wherein the dispersed phase is dispersed within the dispersion medium.

2. The pharmaceutical composition of claim 1, wherein the reducing agent is selected from the group consisting of tiopronin, D-penicilamine, or captopril, and any combination thereof.

3. The pharmaceutical composition of claim 2, wherein the reducing agent is tiopronin.

4. The pharmaceutical composition of claim 3, wherein the concentration of tiopronin in the composition is between about 1 mass % and about 5 mass %.

5. The pharmaceutical composition of claim 1, wherein the anhydrous dispersion medium comprises at least one vegetable oil or at least one medium chain triglyceride, or any combination thereof.

6. The pharmaceutical composition of claim 5, wherein the vegetable oil is selected from the group consisting of castor oil, soybean oil, coconut oil, avocado oil, olive oil, almond oil, and any combination thereof.

7. The pharmaceutical composition of claim 5, wherein the medium chain triglyceride is a triglyceride having at least two of the three fatty acid moieties that are derived from saturated open-chain acids having between 6 and 12 carbon atoms.

8. The pharmaceutical composition of claim 7, wherein the saturated open-chain acids are selected from the group consisting of caprylic acid and caproic acid.

9. The pharmaceutical composition of claim 1, wherein the surfactant or solubilizing and suspending agent is selected from the group consisting of non-ionic polyoxyethlene-polyoxypropylene block copolymers, a water-soluble derivative of cellulose, optionally partially cross-linked polyacrylates, polyoxyethylene sorbitan monolaurates, glyceryl distearate, triglycerol monooleate, glyceryl isostearate, polyoxyethylene sorbitan monopalmitates, polyoxyethylene sorbitan monostearates, and polyoxyethylene sorbitan monooleates.

10. The pharmaceutical composition of claim 9, wherein the non-ionic polyoxyethlene-polyoxypropylene block copolymer is poly(ethylene glycol)-block-poly(propylene glycol)-block-poly(ethylene glycol).

11. The pharmaceutical composition of claim 9, wherein the water-soluble derivative of cellulose is selected from the group consisting of carboxymethyl cellulose, methyl cellulose, hydroxyethyl cellulose, and hydroxypropyl cellulose.

12. The pharmaceutical composition of claim 9, wherein the solubilizing and suspending agent is polyoxyethylene (20) sorbitan monooleate.

13. The pharmaceutical composition of claim 1, further comprising at least one taste modifier selected from the group consisting of sweeteners, flavoring agents, and anesthetic agents.

14. A method for treating, preventing or alleviating a disease, condition, syndrome, symptom, pathology, or malady in a mammalian subject in need of such treatment comprising orally administering to the subject the composition of claim 1.

15. The method of claim 14, wherein the disease being prevented or treated is selected from the group consisting of kidney stone disease, cystinuria, bladder stone disease, ureter stone disease, rheumatoid arthritis and mucus formation in the airways, lungs, bronchi, and trachea of a patient.

16. The method of claim 15, wherein the disease being prevented or treated is cystinuria.

17. The pharmaceutical composition of claim 1, wherein the dispersed phase further comprises a therapeutically effective quantity of at least one urine alkanizing agent.

18. The pharmaceutical composition of claim 17, wherein the urine alkanizing agent is selected from the group consisting of alkali metal salts of citric acid, alkaline-earth metal salts of citric acid, and sodium bicarbonate.

19. The pharmaceutical composition of claim 18, wherein the alkali or alkaline-earth metal salts of citric acid are selected from the group consisting of potassium citrate, sodium citrate, and magnesium citrate.

20. The pharmaceutical composition of claim 17, wherein the concentration of the urine alkanizing agent in the composition is between about 2 mass % and about 20 mass %.

21. The pharmaceutical composition of claim 17, wherein the formulation is adapted for oral administration.
